(19) United States
(12) Patent Application Publication
(10) Pub. No.: US 2017/0020631 A1
(43) Pub. Date: Jan. 26, 2017

(54) Blood Collection Wristband

(71) Applicant: MetaMedical, LLC, Des Moines, IA (US)

(72) Inventor: Auston Horras, Des Moines, IA (US)

(21) Appl. No.: 15/215,733

(22) Filed: Jul. 21, 2016

Related U.S. Application Data
(60) Provisional application No. 62/195,888, filed on Jul. 23, 2015.

(51) Int. Cl. A61B 90/53; A61B 5/15; A61B 5/153

(52) U.S. Cl. CPC A61B 90/53; A61B 5/153; A61B 5/150259; A61B 5/150992; A61B 5/150343

(57) ABSTRACT

A blood collection device includes a wristband with a base and a main body. The main body includes a plurality of tube openings that are sized and shaped to receive and hold blood tubes, and a hub opening that is sized and shaped to receive a hub. The hub opening includes a slot that allows a tube connected to the hub to be fed through the slot thereby obviating the need to fish the needle and butterfly through the entire length of the hub opening. This improves the safety of using a hub with an attached needle. In addition, by placing the hub and the blood tubes in a convenient place for the phlebologist, this further improves safety, speeds the blood drawing process and reduces the potential for work place injuries.

### CROSS REFERENCE TO PRIOR APPLICATION

This patent claims priority from U.S. Provisional Application No. 62/195,888 filed on Jul. 23, 2015.

### FIELD OF THE INVENTION

This invention relates generally to a wristband. More specifically, and without limitation, this invention relates to a wristband used in the blood collection process by a phlebotomist.

### BACKGROUND OF INVENTION

Phlebotomy is the process of making an incision in a vein with a needle for the purposes of drawing blood from the patient. The procedure itself is known as a venipuncture.

Phlebotomists are trained to draw blood from a patient for clinical or medical testing, transfusions, donations, or research. Phlebotomists collect blood primarily by performing venipunctures.

Blood is often drawn through the use of a syringe having a hypodermic needle affixed to an end of a hollow barrel having a plunger slidably positioned within the barrel. Once the needle is inserted into a vein of a patient, the plunger within the hollow barrel is withdrawn causing a vacuum thereby drawing blood into the space created between the tip of the plunger and the barrel.

While using a syringe with a hypodermic needle is useful for drawing a small amount of blood, as medical testing technology has improved and become more complicated so has the quantity of blood needed to perform various tests. As such, the quantity of blood needed often exceeds the quantity of blood that can be withdrawn by a single syringe. In addition, as medical testing technology has become more sophisticated, and more tests have been developed a need has developed to draw blood into a plurality of separate containers, each needed for their own separate tests.

To avoid the displeasure of sticking a patient several times with different syringes, various systems have been developed where a single hub with a needle affixed to the hub is used to fill any number of vials with only a single needle insertion. One such system is known as the Vacutainer® system manufactured by Becton Dickinson headquartered in Franklin Lakes, N.J.

The Vacutainer® system provides many advantages, one of which is that several blood tubes can be filled using a hub and a needle that is inserted into the patient. This eliminates the pain associated with multiple needle insertions, speeds the process of collecting a large volume of blood, and allows blood to be collected in different blood tubes so that each can have their own additives for different tests, among many other advantages.

While the Vacutainer® system provides many advantages, it provides a number of challenges as well. Namely, the Vacutainer® system requires a needle to be inserted into the patient with a short tube connecting the needle to a hub. When inserted, this hub must be held in place close to the point of insertion, otherwise the needle will be pulled out of the patient. This requires the phlebologist to hold the hub in place. Simultaneously, while the hub is held in place, the phlebologist must insert empty blood tubes into the hub, and remove the blood tubes once filled. This insertion and withdrawal process is difficult to do when the phlebologist must maintain one hand on the hub near the insertion point. In addition, this process is complicated when there are a plurality of blood tubes that must be used as these blood tubes must be placed somewhere before, during and after the blood drawing process. If handled improperly, these blood tubes can be dropped, mixed-up, damaged, lost, discarded or misplaced. In addition, when there is no convenient location to place the blood tubes during the blood drawing process, this can cause the phlebologist to reach across their bodies, or twist in unusual ways, which often causes repetitive use or motion injuries, which is a leading cause of work-place injuries for phlebologists. In addition, when there is no convenient location to place blood tubes during the blood drawing process, this may lead the phlebologist to place, hold or store the blood tubes in an unsafe or unsanitary manner such as in their mouth, in a pocket or really any other convenient, but unsafe or unsanitary place or manner.

To address these deficiencies, a number of devices have been developed. One such device is presented in U.S. Pat. No. 5,749,504 to Bieker entitled Blood Collection Tube Holder. While this system provides a wrist strap that holds a number of blood tubes, this system suffers from a number of disadvantages. Namely, this system does not provide the ability to hold both a hub as well as blood tubes. In addition, the flexible nature of the holders means in practice to ensure the blood tubes are properly held in the holders, excessive force must be applied upon insertion. This means that excessive force must be applied to remove the blood tubes from the flexible holders. This can cause a dangerous situation, and can cause the device to rotate upon the phlebologist's wrist upon insertion and removal, which is dangerous, can be painful and can itself cause a repetitive use injury, and can cause blood tubes to be dropped, dislodged or even opened accidently. As such, the Bieker system suffers from a number of substantial disadvantages.

Another device is presented in U.S. Pat. No. 7,255,251 to Smith entitled Holding Appliance For Facilitating Blood Drawing Process. While this system provides a wrist strap that holds a hub, this system does not hold any blood tubes. As such, when using this device with a number of blood tubes, the blood tubes must be carried separately, which have a number of disadvantages associated therewith, as is addressed above.

Therefore, for the reasons stated above, and for other reasons stated below which will become apparent to those skilled in the art upon reading and understanding the specification, claims and drawings, there is a need in the art for an improved blood collection wristband. Thus, an object of the invention is to provide a blood collection wristband that improves upon the present state of the art.

Another object of the invention is to provide an improved blood collection wristband system that is simple to use.

Yet another object of the invention is to provide an improved blood collection wristband system that is easy to use.

Another object of the invention is to provide an improved blood collection wristband system that that is relatively inexpensive to manufacture.

Yet another object of the invention is to provide an improved blood collection wristband system that improves safety.

Another object of the invention is to provide an improved blood collection wristband system that reduces repetitive use injuries.

Yet another object of the invention is to provide an improved blood collection wristband system that conveniently places a hub and a plurality of blood tubes in a single location.

Another object of the invention is to provide an improved blood collection wristband system that is comfortable to wear.

Yet another object of the invention is to provide an improved blood collection wristband system that holds hubs and blood tubes from various manufacturers.

Another object of the invention is to provide an improved blood collection wristband system that allows for a butterfly to be removed from the device in a safe and convenient manner.

Yet another object of the invention is to provide an improved blood collection wristband system that is formed of a minimum number of parts.

Another object of the invention is to provide an improved blood collection wristband system that fits any phlebologist.

Yet another object of the invention is to provide an improved blood collection wristband system that provides increased convenience.

Another object of the invention is to provide an improved blood collection wristband system that reduces the amount of time needed to draw blood.

Yet another object of the invention is to provide an improved blood collection wristband system that increases patient comfort.

Another object of the invention is to provide an improved blood collection wristband system that provides a level of reassurance to the patient.

Yet another object of the invention is to provide an improved blood collection wristband system that has a robust design.

Another object of the invention is to provide an improved blood collection wristband system that allows for quick and easy insertion and withdrawal of blood tubes and hubs.

Yet another object of the invention is to provide an improved blood collection wristband system that provides the phlebologist with a free hand during the blood drawing process.

Another object of the invention is to provide an improved blood collection wristband system that is durable.

Yet another object of the invention is to provide an improved blood collection wristband system that is sanitary.

Another object of the invention is to provide an improved blood collection wristband system that has a long useful life.

Yet another object of the invention is to provide an improved blood collection wristband system that has an intuitive design.

These and other objects, features, or advantages of the invention will become apparent from the specification and claims.

### SUMMARY OF THE INVENTION

A blood collection device includes a wristband with a base and a main body. The main body includes a plurality of tube openings that are sized and shaped to receive and hold blood tubes, and a hub opening that is sized and shaped to receive a hub. The hub opening includes a slot that allows a tube connected to the hub to be fed through the slot thereby obviating the need to fish the needle and butterfly through the entire length of the hub opening. This improves the safety of using a hub with an attached needle. In addition, by placing the hub and the blood tubes in a convenient place for the phlebologist, this further improves safety, speeds the blood drawing process and reduces the potential for work place injuries.

### BRIEF DESCRIPTION OF THE DRAWINGS

FIG. 1 is a perspective view of a blood collection system shown in use on the wrist of a phlebologist, the view predominantly showing the front side of the blood collection system having a base and a main body, the base having a wristband connected thereto that is wrapped around the wrist of a phlebologist and adhered to itself with a hook and loop arrangement, the main body having a centrally positioned hub opening with a hub positioned therein, the main body having a pair of tube openings positioned on either side of the hub opening with blood tubes positioned therein, the hub having a barrel, a nipple, a tube a butterfly, a shield and a cover;

FIG. 2 is another perspective view of the blood collection system shown in FIG. 1, the view showing the blood collection system removed from the wrist of a phlebologist;

FIG. 3 is another perspective view of the blood collection system shown in FIGS. 1 and 2, the view showing the blood collection system removed from the wrist of a phlebologist;

FIG. 4 is another perspective view of the blood collection system shown in FIGS. 1, 2 and 3, the view showing the blood collection system in an exploded arrangement with the hub and blood tubes removed from the main body;

FIG. 5 is a top elevation view of a blood collection system having three tube openings on either side of the hub opening;

FIG. 6 is a bottom elevation view of the blood collection system shown in FIG. 5, the view showing the base connected to the main body;

FIG. 7 is a side elevation view of the blood collection system shown in FIGS. 5 and 6, the view showing the base connected to the main body;

FIG. 8 is a front side elevation view of the blood collection system shown in FIGS. 5, 6 and 7, the view showing the three tube openings positioned on either side of the generally centrally positioned hub opening, the view showing the slot that provides access to the hub opening;

FIG. 9 is a rear side elevation view of the blood collection system shown in FIGS. 5, 6, 7 and 8, the view showing the three tube openings positioned on either side of the generally centrally positioned hub opening, the view showing the slot that provides access to the hub opening;

FIG. 10 is a perspective view of the blood collection system shown in FIGS. 5, 6, 7, 8 and 9, the view showing the three tube openings positioned on either side of the generally centrally positioned hub opening, the view showing the slot that provides access to the hub opening;

FIG. 11 is a another perspective view of the blood collection system shown in FIGS. 5, 6, 7, 8, 9 and 10, the view showing the three tube openings positioned on either side of the generally centrally positioned hub opening, the view showing the slot that provides access to the hub opening;

FIG. 12 is a perspective view of the blood collection system shown in FIGS. 5, 6, 7, 8, 9, 10 and 11, the view showing an arrangement wherein the base is a separate component from the main body, the view showing the base exploded from the main body.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that mechanical, procedural, and other changes may be made without departing from the spirit and scope of the invention(s). The following detailed description is, therefore, not to be taken in a limiting sense, and the scope of the invention is defined only by the appended claims, along with the full scope of equivalents to which such claims are entitled.

As used herein, the terminology such as vertical, horizontal, top, bottom, front, back, end and sides are referenced according to the views presented and the orientation and arrangement of parts. It should be understood, however, that the terms are used only for purposes of description, and are not intended to be used as limitations. Accordingly, orientation of an object or a combination of objects may change without departing from the scope of the invention.

With reference to the figures, blood collection system 10 (system 10) is presented herein. The system 10 is formed of any suitable size, shape and design and is configured to assist a phlebologist in the blood collection process.

In one arrangement, the system 10 includes a wristband 12 with a holder 14 connected thereto. Holder 14 is configured to hold a plurality of blood tubes 16 therein. The blood tubes 16 include a vial section 18 which is covered by a cap 20. The holder 14 is also configured to hold a hub 22 therein. In one arrangement, the hub 22 includes a barrel 24, a nipple 26 connected to the barrel 24, a tube 28 connected to the nipple 26, a needle 30 having a butterfly 32 connected to the tube 28, the needle 30 is covered by a removable cover 34, and a shield 36 slidably positioned over a portion of the tube 28, needle 30 and butterfly 32 to cover the needle 30 after use.

Wristband 12 is formed of any suitable size, shape and design and serves to connect holder 14 to a phlebologist's wrist in a comfortable manner. In the arrangement shown, wristband 12 is formed of a length of material and extends from a first end 38 to a second end 40. Wristband 12 is formed of any flexible material that is comfortable to wear and is formed of a suitable width to ensure the wristband 12 remains on the phlebologist's wrist and provides adequate support for holder 14 during use and helps to maintain the position and orientation of the holder 14. In one arrangement, a width of approximately 1.5 inches has been tested with success, however any width is hereby contemplated for use. In one arrangement, a heavy-duty woven nylon-type material has been tested with success, however any other material is hereby contemplated for use.

In the arrangement shown, a buckle 42 is connected to the first end 38 of wristband 12. Buckle 42 is formed of any suitable size, shape and design and serves to allow the wristband 12 to fold over and connect to itself, as well as serving to allow the length of wristband 12 to be easily adjusted. In the arrangement shown, buckle 42 is generally rectangular in shape and is slightly wider than the width of wristband 12 and has an open interior that allows the passage of the second end 40 of the wristband 12 there through. In one arrangement, wristband 12 is attached to buckle 42 by passing a length of the wristband 12 adjacent the first end 38 through the open interior of buckle 42 and folding it back on itself Once folded over, the wristband material is attached to itself by any method, manner or means such as welding, gluing, stitching or the like. In this way, buckle 42 is attached to first end 30 of wristband 12.

In one arrangement, as is shown, wristband 12 is connected to itself and held in place using a hook and loop arrangement, such as Velcro® or the like. In this arrangement, as is shown, a hook section 44 is connected to one side of wristband 12 adjacent second end 40 and extends a length toward first end 38, and a loop section 46 is positioned adjacent the inward end of the hook section 44, toward first end 38 and extends inward a length of wristband 12, or extends the entire length, or most of the length, from the inward edge of the hook section 44 to the first end 38. Any other arrangement of a hook and loop arrangement is hereby contemplated for use for connecting the wristband 12 to the phlebologist's wrist. Alternatively, an elastic or stretchable device is used as wristband 12. Alternatively, a drawstring type system is used to tighten wristband 12. Alternatively, a belt and buckle arrangement is used to tighten wristband 12. Alternatively, an arrangement of buttons or snaps is used to fix wristband 12 onto the phlebologist's wrist. Alternatively, any other manner, method or combination thereof is hereby contemplated for use to tighten the wristband 12 on to the phlebologist's wrist.

In one arrangement, a label 50 is connected to wristband on a side opposite hook section 44 and loop section 46. Label 50 is any form of an indicia that can identify information about the system 10, such as the name of the device, the manufacturer, the manufacturer's address or contact information or any other information about the manufacturer, or the label 50 contains information about the phlebologist, the hospital, the tests that are being performed, or any other relevant information. Alternatively, the label 50 can have or include credentials about the phlebologist, such as a license, a certification card, an ID or any other credential that can provide a level of comfort and reassurance to the patient during use. In an alternative arrangement, instead of a label 50 or in addition to a label 50, the wristband includes a receptacle 51, that is configured to hold identification such as a license card, a picture ID, an ID, a picture, or any other object that can be used to identify the phlebologist, the tests that are to be performed using the system 10, or any other information. In one arrangement, receptacle 51 is a clear plastic holder, similar to that used in many purses and wallets that is configured to hold an ID therein.

Label 50 and/or receptacle 51 are placed on the wristband 12 on a side opposite the hook section 44 at or near the second end 40. Placement at or near the second end ensures that the label 50 and/or receptacle 51 is/are visible as it is this portion of the wristband that passes through buckle 42 and is folded back on itself thereby holding wristband on the phlebologist's wrist. As such, it is this portion of wristband 12 that is outwardly visible when system 10 is worn.

Holder 14 is formed of any suitable size, shape and design and serves to hold a plurality of blood tubes 16 and a hub 22 therein. In the arrangement shown, holder 14 includes a base 54 and a main body 56. In one arrangement, base 54 and main body 56 are formed of a single unitary piece such as through injection molding, casting, machining or the like that results in a single piece construction. In an alternative arrangement, the base 54 and main body 56 are formed of separate pieces that are then joined together through a secondary manufacturing step such as by gluing, welding, screwing, bolting, a snap-fit arrangement, a friction-fit arrangement, or the like or any combination thereof. Alternatively, the base 54 and main body 56 are formed of any number of or plurality of pieces.

Base 54 is formed of any suitable size, shape and design and serves to connect to the wristband 12, and to connect the holder 14 to the wristband 12. In the arrangement shown, as one example, base 54 has a lower surface 58 that has a generally flat center portion 60 with arcuate side portions 62 that curve or angle downward as they extend away from the center portion 60. This angled or curved arrangement of side portions 62 is configured to comfortably fit the curvature of a phlebologist's wrist. Base 54 includes an upper surface 64 having arcuate side portions 68 that curve or angle downward as they extend away from the center of the base 54 so as to generally follow the lower surface 58 in arcuate spaced relation.

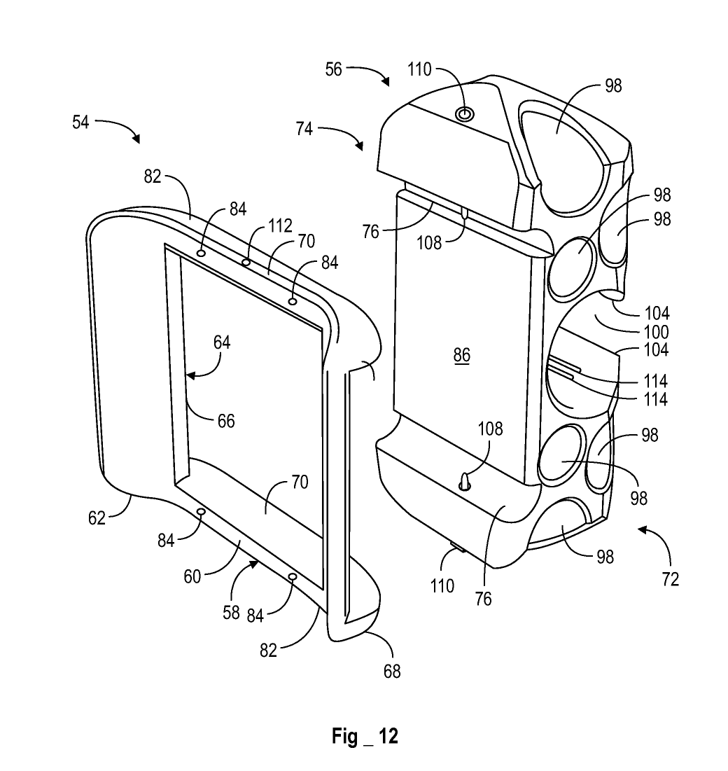

In one arrangement, the base 54 and main body 56 are formed of a single unitary piece. In an alternative arrangement, as is shown in FIG. 12, the base 54 are formed of separate pieces which are joined together by any manner, method or means, such as by gluing, welding, screwing, bolting, a snap-fit arrangement, a friction-fit arrangement, or the like. In the arrangement shown in FIG. 12, base 54 includes cross braces 70 which extend between the front side 72 of main body 56 and the rear side 74 of the main body 56. In the arrangement shown, these cross braces 70 are generally rectangular in shape and are elongated, however any other size, shape or design is hereby contemplated for use.

These cross braces 70 are sized and shaped to fit within recesses 75 in the bottom side of main body 56 thereby facilitating a strong, durable and secure connection between the two components. These recesses 75 extend from the front side 74 of the main body 56 to the rear side 74 of the main body 56 and facilitate nesting of the two components to provide a low-profile connection there between.

A slot 80 is positioned between side portions 62, 68 that serves to allow wristband 12 to pass there through thereby connecting base 54 to wristband 12 and to allow the position of base 54 to be adjusted on wristband 12. Slots 80 extend a distance between the sides 82 of base 54. In the arrangement where base 54 and main body 56 are separate pieces, base 54 includes one or more holes 84 that allow the passage of a fastener, such as a screw, bolt, or the like through base 54 and into the bottom surface main body 56 thereby connecting main body 56 to base 54. When the base 54 and main body 56 are formed of a single piece, fasteners are not needed.

Main body 56 is formed of any suitable size, shape and design and serves to hold a hub 22 and one or more blood tubes 16 in a convenient position for a phlebologist's use during a blood drawing process. In one arrangement, main body 56 has a bottom side 86 and a top side 88 that are connected by ends 90. Main body 56 also includes opposing walls 92 that define the front side 72 and back side 74 of main body 56. In the arrangement shown, main body 56 has a generally flat bottom side 86. Bottom side 86 arcuately curves at ends 90 upward toward top side 88. Having ends 90 curve smoothly and create a smooth transition, instead of sharp corners, between bottom side 86 and top side 88 helps to prevent the main body 56 from getting caught up on clothing or other objects when system 10 is worn by a phlebologist. Walls 92 are generally flat, square and extend in generally parallel spaced relation save for that the walls 92 tend to taper inward as they extend upward toward top side 88 in a smooth and arcuate fashion, again so as to prevent system 10 from getting caught on clothing or other objects during use. That is the main body 54, as well as base 54, tend to have smooth and rounded corners in an attempt to avoid sharp corners or edges that can get caught on objects.

Main body 56 includes a plurality of tube openings 98 that are sized and shaped to receive blood tubes 16, and a hub opening 100, that is sized and shaped to receive a hub 22. In the arrangement shown, hub opening 100 is generally centrally positioned between ends 90 and extends in a generally perpendicular manner through the planes formed by opposing walls 92. Hub opening 100, when viewed from one end or the other, has a generally circular shape, however hub opening 100 slightly narrows as it extends from one wall 92 to the other wall 92 so as to cause a friction-fit with hub 22 when hub 22 is inserted within the hub opening 100. The narrower end of hub opening 100 terminates in a collar 102 that steps in, or is narrower than the remaining portions of hub opening 100. Collar 102 serves to engage the end of barrel 24 of hub 22, so as to prevent hub 22 from sliding through hub opening 100, while providing enough clearance for nipple 26 and tube 28 to extend there through. In the arrangement shown, collar 102 is positioned near to or adjacent to the back side 74 of main body 56.

A slot 104 is positioned in the upper portion of hub opening 100. Slot 104 extends from the front side 72 of main body 56 to the back side 74 of main body 56. Or, said another way, slot 104 extends from wall 92 to wall 92. Slot 104 allows vertical access into hub opening 100.

Hub 22 is inserted into hub opening 100 from front side 74 of wall 92 until the end of barrel 24 engages collar 102, or until the narrowing or tapered nature of hub opening 100 frictionally engages the barrel 24 of hub 22 thereby stopping the inward progression of hub 22 into hub opening 100. Slot 104 allows tube 28 to be dropped into or removed from hub opening 100 without the need to fish the entire length of tube 28 through hub opening 100. Slot 104 is a safety feature as it allows tube 28 to be quickly and easily removed from hub opening 100. This is important because needle 30 is attached to the end of tube 28, which is a safety hazard when handled improperly. In addition, most hub 22 and needle 30 combinations include a butterfly 32 which consists of a pair of wings that extend outward from the needle 30. Butterfly 32 assists the phlebologist with alignment and insertion of the needle 30 into the patient. However, the width 32 of most butterflies 32 is substantially greater than the width of the hub opening 100. As such, the slot 104 in the upper surface of hub opening 100 allows for the insertion and withdrawal of the hub 22 into hub opening 100 without the need to awkwardly force butterfly 32 through hub opening 100. Again, this increases safety as butterfly 32 is connected to needle 30.

To assist with the insertion of tube 28 into hub opening 100 through slot 104, a pair of panels 106 angle downward from top side 88 of main body 56 until they connect with slot 104. This downward angling of panels 106 helps to provide alignment of tube 28 and urges tube 28 to slide into hub opening 100 through slot 104. As such, the angled panels 106 increases ease of use, as well as improves safety, and reduces the amount of time it takes to insert a hub 22 into hub opening 100.

In a similar fashion, the arcuate, or generally circular nature of hub opening 100 serves the similar function when a phlebologist goes to remove tube 28 from hub opening 100. Upon removal, the arcuate sidewalls of hub opening 100 tend to urge the tube 28 toward the center of hub opening 100 and out of slot 104 when the hub 22 is dislodged from a friction fit with hub opening 100 and the tube 28 is raised. This naturally provides a quick, easy and safe removal process.

In the arrangement shown, main body 56 includes a plurality of tube openings 98. Any number of tube openings 98 are hereby contemplated for use such as one, two, three, four, five, six, seven, eight, nine, ten or more. In the arrangement shown, six tube openings 98 are presented, three on either side of hub opening 100. In the arrangement shown, a first tube opening 98 is positioned adjacent hub opening 100 and near the top side 88 of main body 56. In the arrangement shown, this tube opening 98 is a large-size tube opening 98 configured to receive a large size blood tube 16. In the arrangement shown, a second tube opening 98 is positioned adjacent hub opening 100 and near the bottom side 60/86 of main body 56. In the arrangement shown, this tube opening 98 is a small-size tube opening 98 configured to receive a small size blood tube 16. In the arrangement shown, a third tube opening 98 is positioned outward from the first and second tube openings 98 which are adjacent hub opening 100. In the arrangement shown, this tube opening 98 is a large-size tube opening 98 configured to receive a large size blood tube 16.

Tube openings 98 extend in a generally perpendicular manner through the planes formed by opposing walls 92. Tube openings 98, when viewed from an end, have a generally circular shape, however tube openings 98 slightly narrow as they extend from the wall 92 of the front side 72 to the wall 92 of the back side 74. This narrowing of tube openings 98 as they extend from the front side 72 to the back side 74 cause a friction-fit with blood tubes 16 when inserted therein. The narrower end of tube openings 98 are positioned on the same side that the hub opening 100 terminates in a collar 102. This ensures that hub 22 and blood tubes 16 are inserted into and withdrawn from the same wall 92 of main body 56. As there is no tube, needle, butterfly or other object connected to blood tubes 16 (other than cap 20) there is no need for a slot in tube openings 98 as there is with hub opening 100.

While the sizes of blood tubes 16 are generally standardized, there are two common sizes, a small size (13 mm diameter which holds between 2 to 4 mL) and a large size (16 mm diameter which holds between 6 to 12 mL). In one arrangement, tube openings 98 are sized and shaped to accommodate both the large and small sized blood tubes 16. In another arrangement, some of the tube openings 98 are specifically sized for the large blood tubes 16 and others of the tube openings 98 are specifically sized for the small blood tubes 16.

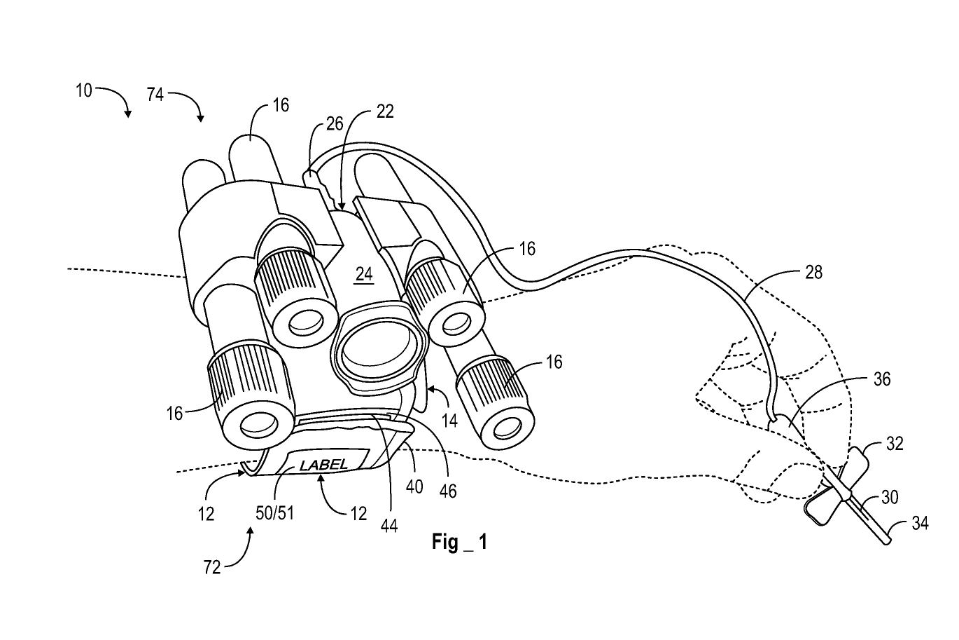

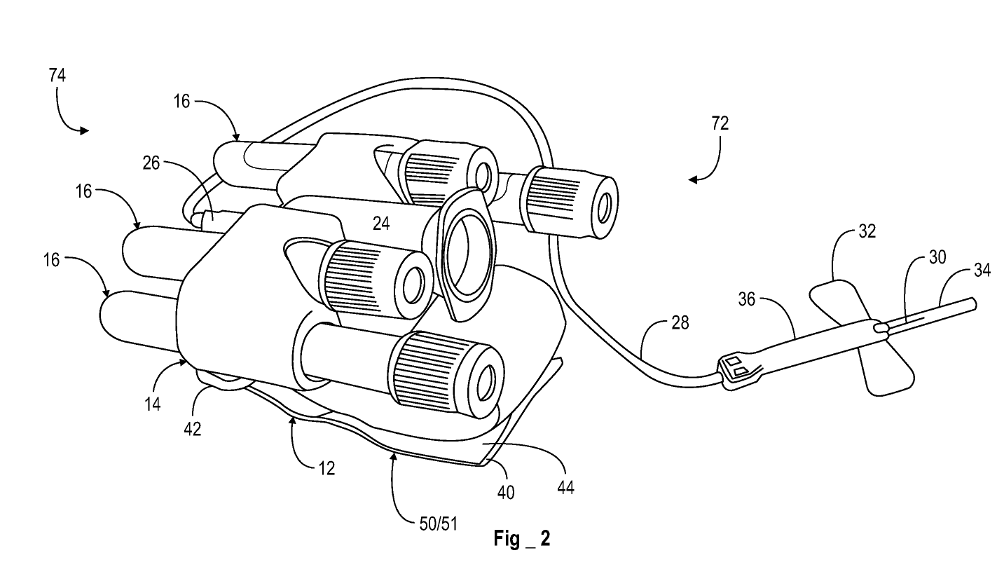

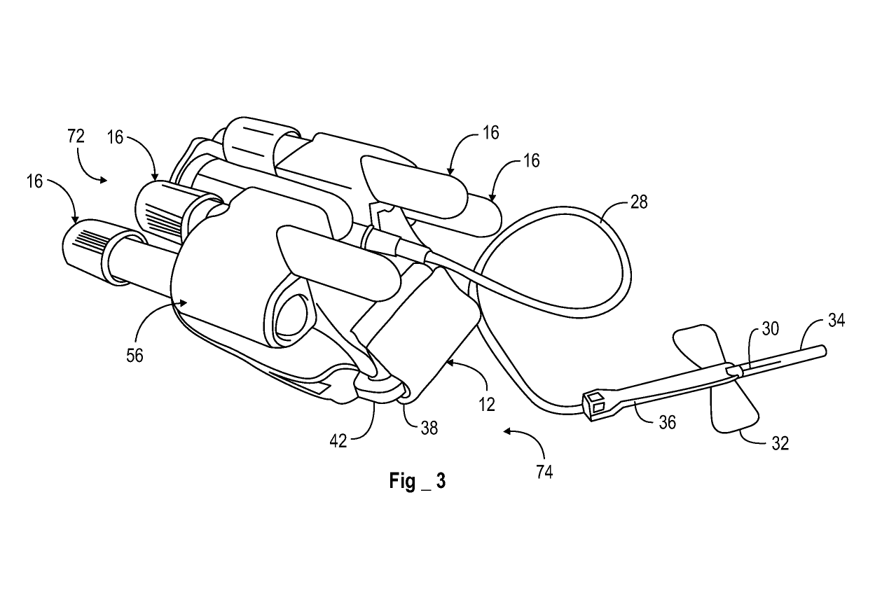

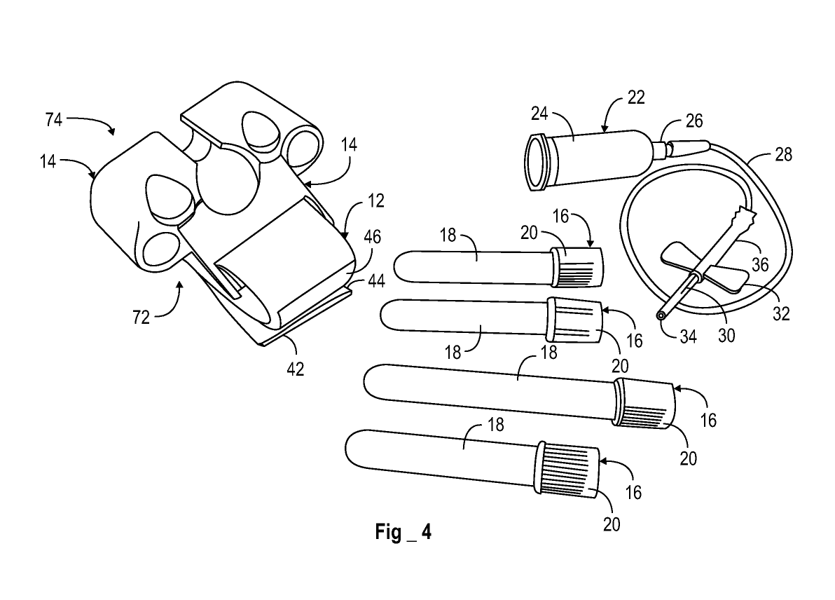

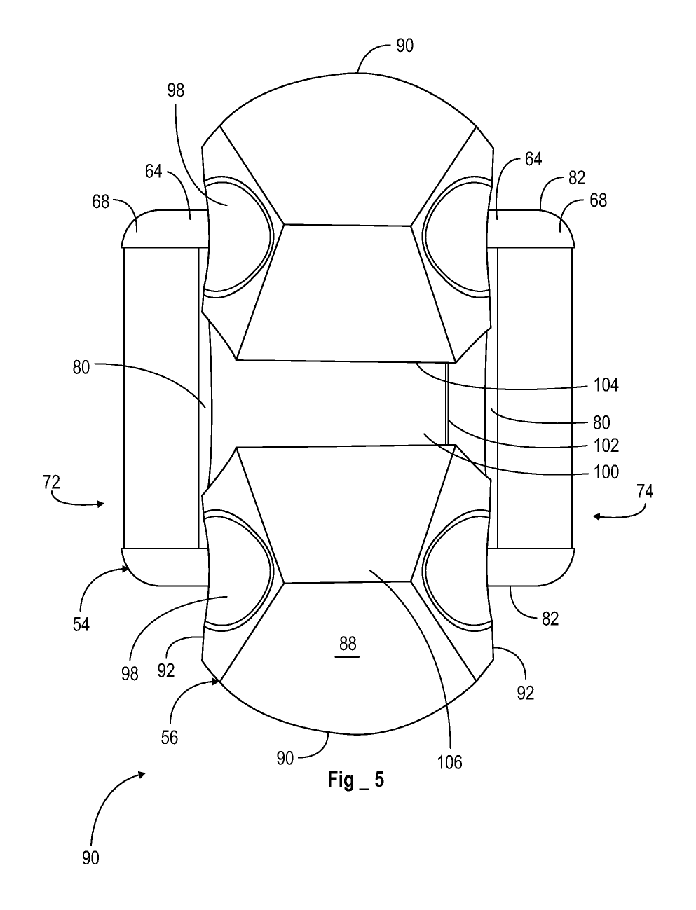

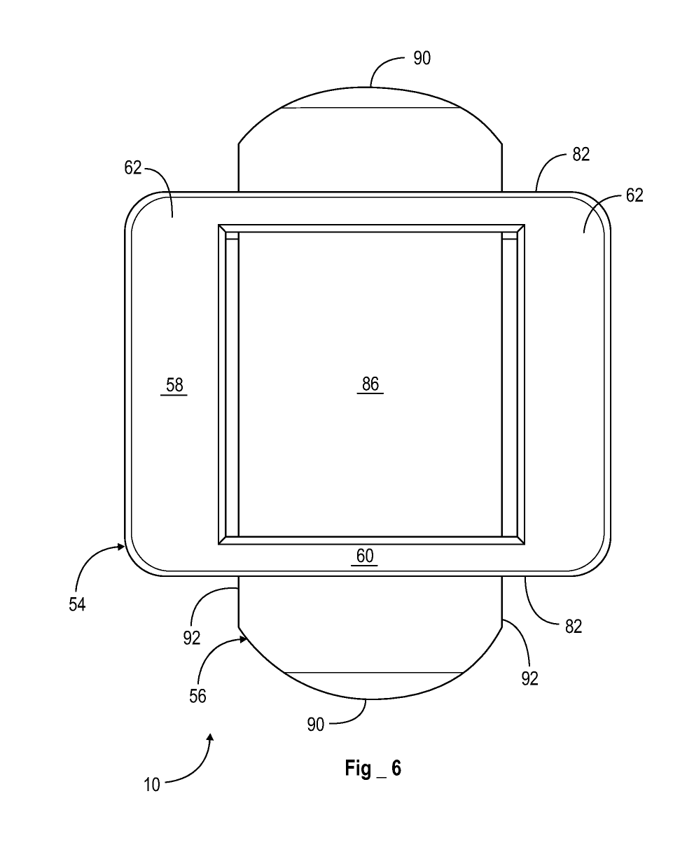

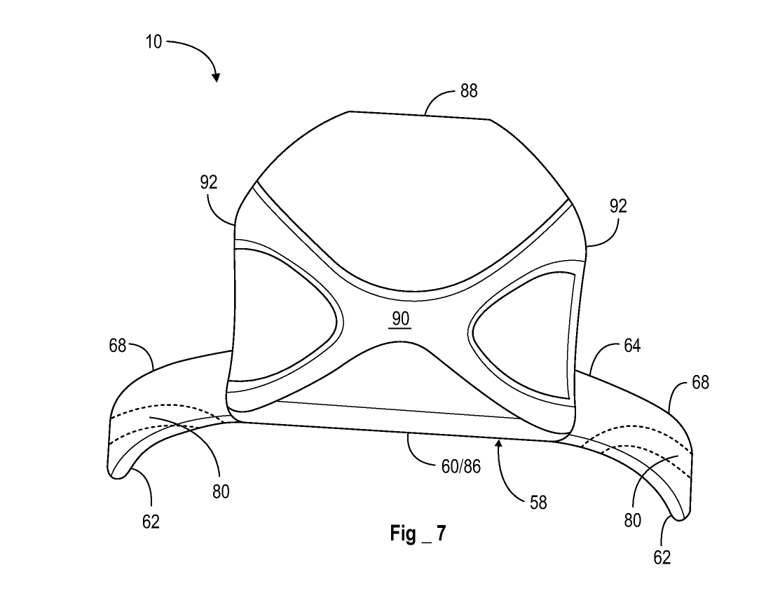

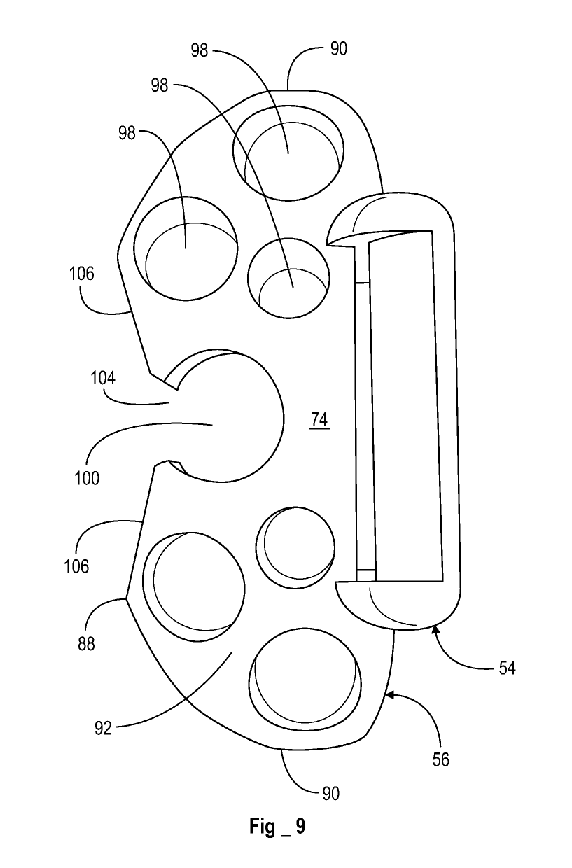

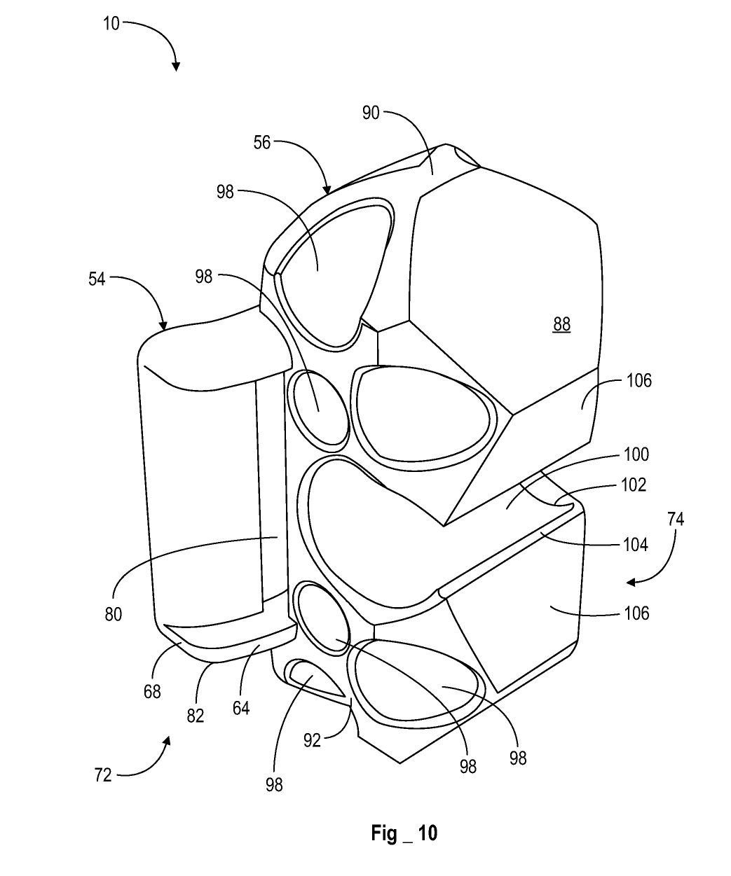

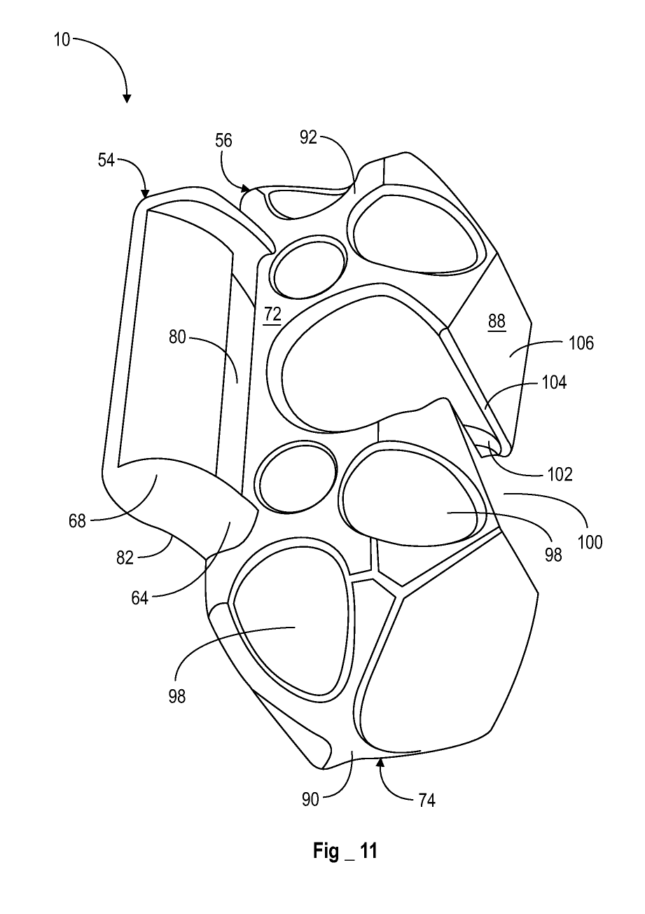

Potential Configurations: Any combination of hub opening 100 and tube openings 98 are hereby contemplated for use. As examples, the following are contemplated configurations for main body
Hub Only:
   Hub opening 100 only.
Small Tube Openings:
   Hub opening 100, single small tube opening 98.
   Hub opening 100, two small tube openings 98, one on either side of hub opening 100.
   Hub opening 100, three small tube openings 98, two on one side of hub opening 100 and one on the other side of hub opening 100.
   Hub opening 100, four small tube openings 98, two on either side of hub opening 100.
   Hub opening 100, five small tube openings 98, three on one side of hub opening 100 and two on the other side of hub opening 100.
   Hub opening 100, six small tube openings 98, three on either side of hub opening 100.
Large Tube Openings:
   Hub opening 100, single large tube opening 98.
   Hub opening 100, two large tube openings 98, one on either side of hub opening 100.
   Hub opening 100, three large tube openings 98, two on one side of hub opening 100 and one on the other side of hub opening 100.
   Hub opening 100, four large tube openings 98, two on either side of hub opening 100.
   Hub opening 100, five large tube openings 98, three on one side of hub opening 100 and two on the other side of hub opening 100.
   Hub opening 100, six large tube openings 98, three on either side of hub opening 100.
Combination of Small and Large Tube Openings
   Hub opening 100, one small tube openings 98, and one large tube opening 98, one on either side of hub opening 100.
   Hub opening 100, two small tube openings 98, and one large tube opening 98 with two small tube openings 98 on one side of hub opening 100 and one large tube opening 98 on the other side of hub opening 100.
   Hub opening 100, two small tube openings 98, and one large tube opening 98 with one small tube openings 98 on either side of hub opening 100 and one large tube opening 98 on one side of hub opening 100.
   Hub opening 100, two small tube openings 98, and two large tube openings 98, one small tube opening on either side of the hub opening 100 and one large tube opening 98 on either side of hub opening 100.
   Hub opening 100, two small tube openings 98, and two large tube openings 98, two small tube opening on one side of the hub opening 100 and two large tube openings 98 on the other side of hub opening 100.
   Hub opening 100, three small tube openings 98, and one large tube openings 98, two small tube opening on one side of the hub opening 100 and one large tube opening 98 and one small tube opening 98 on the other side of hub opening 100.
   Hub opening 100, three small tube openings 98, and one large tube openings 98, three small tube openings on one side of the hub opening 100 and one large tube opening 98 on the other side of hub opening 100.
   Hub opening 100, two small tube openings 98, and four large tube openings 98, one small tube opening on either side of the hub opening 100 and two large tube opening 98 on either side of hub opening 100.
   Hub opening 100, two large tube openings 98, and small large tube openings 98, one large tube opening on either side of the hub opening 100 and two small tube opening 98 on either side of hub opening 100 (this is the arrangement shown in FIGS. 1-12).

These are merely some of countless examples and any other combination of small and large tube openings 98 and the number of tube openings 98 is hereby contemplated for use.

As each patient and each situation is different, it is sometimes necessary to use various numbers of small blood tubes 16 and large blood tubes 16. To facilitate these varying needs, in one arrangement, main body 56 is quickly and easily detachable from base 54 by any manner, method or means. This allows wristband 12 and base 54 to be used with a plurality of main bodies 56, each main body 56 having a different number of small and large tube openings 98. This allows the phlebologist to simply detach the currently attached main body 56 and attach the main body 56 that suits the particular patient or situation. The quick detach mechanism may be formed of one or more spring loaded pins, one or more deflectable arms with hooks, a button activated spring loaded attachment device, or any other manner, method or means of quickly and easily attaching and detaching two objects.

In one arrangement, as is shown in FIG. 12, a pair of spring loaded pins 108 extend into recesses 76 that receive cross braces 70 of base 54. These spring loaded pins 108 are connected to and operated by buttons 110 that face outward from ends 90 of main body 56. These spring loaded pins 108 are received within openings 112 in the cross braces 70 of base 54. To release a main body 56 from base 54 the buttons 110 are depressed thereby withdrawing the pins 108 from openings 112 in base 54 and the main body 56 is removed. To attach a main body 56 to base 54, the main body 56 is aligned with the base 54, or more specifically the recesses 76 in the main body 56 are aligned to fit over the cross braces 70 of base 54 and the main body 56 is forced onto the base 54. As the main body 56 is forced onto the base 54, the spring loaded pins 108 deflect to allow passage of the cross braces 70 into the recesses 76. Once the main body 56 is fully inserted over the base 54 and the openings 112 are aligned with the pins 108, the spring loaded bias of the pins 108 automatically forces the pins 108 into the openings 112 thereby removably connecting the main body 56 to the base. In this way, the perfect main body 56 having the proper number of tube openings 98 therein, is quickly and easily installed on base 54 for use. Any other manner, method or means of removably connecting main body 56 to base 54 is hereby contemplated for use.

In one arrangement, as is shown, the tube openings 98 and hub openings 100 are generally rigid and therefore depend on generally close and tight tolerances between the openings 98 and hub opening 100 and the blood tubes 16 and hub 22, respectively. In an alternative arrangement, tube openings 98 and/or hub opening 100 are formed of a generally flexible or compressible material or include a generally flexible or compressible piece or object. In one arrangement, a friction member 114, such as a deflectable spring, fin or piece of material is positioned within tube openings 98 and/or hub opening 100 that compresses, bends, deflects or otherwise accommodates blood tube 16 upon insertion and accommodates blood tubes 16 of various sizes. In one arrangement, friction member 114 is a strip of high-friction and/or compressible material such as rubber, compressible plastic, composite, synthetic rubber, or the like that is both compressible and has a high coefficient of friction, that is placed on or in the sidewall of tube openings 98 and/or hub opening 100. In another arrangement, friction member 114 is an O-ring of high-friction and/or compressible material such as rubber, compressible plastic, composite, synthetic rubber, or the like that is both compressible and has a high coefficient of friction, that is placed on or in the sidewall of tube openings 98 and/or hub opening 100. Friction member 114 helps to hold onto hub 22 and/or blood tubes 116 when in place within tube openings 98 or hub opening 100 respectively, and helps to prevent unintentional dislodgement of the hub 22 or blood tubes 16. Friction members 114 also help to accommodate size variations in hub 22 or blood tubes 16.

In an alternative arrangement, instead of tube openings 98 and/or hub openings 100 being formed of the same material as the rest of main body 56, tube openings 98 and/or hub openings 100 include a second material that increases the friction between tube openings 98 and/or hub openings 100 thereby helping to hold blood tubes 16 or hub 22 therein. In this arrangement, the interior surface, or part of the interior surface of hub opening 100 and tube openings 98 are coated with a layer of high-friction and/or compressible material such as rubber, compressible plastic, composite, synthetic rubber, or the like that is both compressible and has a high coefficient of friction.

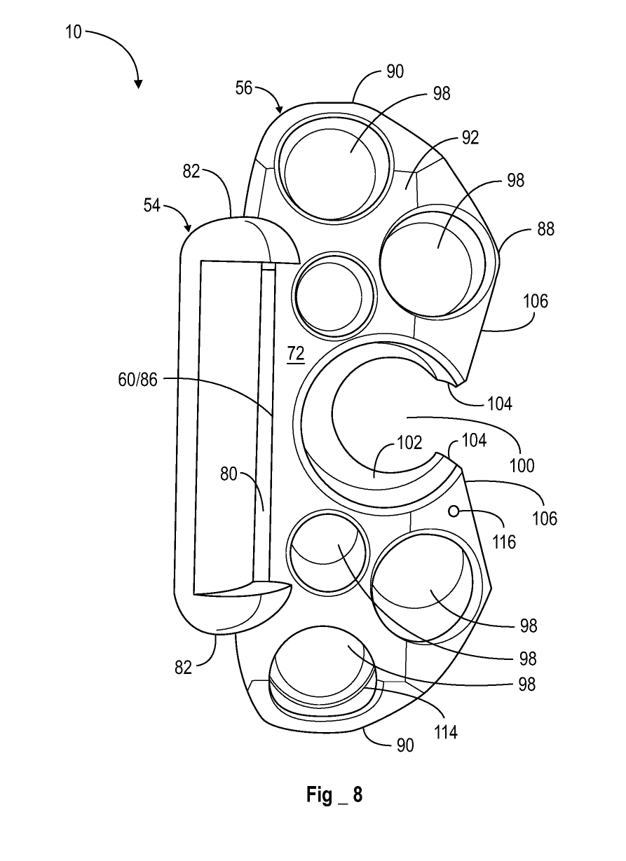

In one arrangement, main body 56 also includes a needle holder 116 therein. Needle holder 116 is formed of any suitable size, shape and design. In one arrangement, as is shown, needle holder 116 is a generally circular or cylindrical opening, much like tube openings 98, that is sized and shaped to hold needle 30 and/or cover 34 and/or shied 36 therein. Because tube 28 is quite long, by providing needle holder 116 in main body 56 and inserting the needle 30 within needle holder 116, this prevents the phlebologist from having to constantly hold the needle 30/butterfly 32 when not in use. This frees up the phlebologist's hand and improves safety by securing the position of the needle 30 at all times. While the needle holder 116 is shown in the front side 72 of main body 56 and extends toward the back side 64 of main body 56 in FIG. 8, the needle holder 116 may be placed in any portion of the main body 56 and in any orientation. As one example, needle holder 116 may be placed in a generally vertical orientation and extend downward into the main body 56 from the top side 88.

In Operation: A phlebologist who needs to draw blood from a patient attaches wristband 12 that extends through slots 80 in base 54 having a main body 56 attached thereto around their wrist. The phlebologist adjusts the length of the wristband 12 to fit their wrist by extending the second end 40 of the wristband 12 through the buckle 42 and folds the hook and loop sections 44, 46 over one another to form a locking engagement. In the arrangement wherein the a receptacle 51 is included for holding an ID therein, the phlebologist inserts their ID within the receptacle 51.

Next, the phlebologist selects the necessary blood tubes 16 and inserts them into the tube openings 98 until they frictionally engage and are frictionally held within the tube openings 98.

Next, the phlebologist selects the appropriate hub 22 and the phlebologist drops the tube 28 through the slot 104 in the hub opening 100. As the tube 28 is dropped into the slot 104 in hub opening 100, the force of gravity and the downwardly angling panels 106 help guide the tube 28 into the hub opening 100. Once the tube 28 is within the hub opening 100, the phlebologist inserts the hub 22 into the hub opening 100 until the barrel 24 frictionally engages and is frictionally held within the hub opening 100 which is likely when the end of barrel 24 engages collar 102. Once fully inserted, the nipple 26 of hub 22 extends outward of the back side 74 of main body 56 adjacent the collar 102 and the tube 28, needle 30, cover 34 and shield 36 are attached thereto.

Once the system 10 is fully assembled with the necessary blood tubes 16 and hub 22, the phlebologist is free to travel to the patient's location without fear of losing or leaving behind any of the blood tubes 16 or hub 22. Once at the patient's location, the phlebologist can hold the butterfly 32, needle 30 and shield 36 in the hand that the system is connected to, and insert the needle 30 into the patient. While holding the needle 30 in place, the phlebologist conveniently uses their other hand to withdraw each blood tube 16 from their respective tube openings 98 and inserts the blood tubes 16 into the hub 22. Once each blood tube is filled, the phlebologist withdraws the blood tubes 16 from the hub 22 and reinserts them into their tube opening 98. This process is repeated until all the blood tubes are filled. The system 10 is rigid enough and the wrist band 12 is wide or strong enough to provide a solid base that allows the phlebologist to insert and remove the blood tubes 16 without the main body 56 substantially moving or twisting on the phlebologist.

System 10 allows the phlebologist to have located in the most convenient location all of the blood tubes 16, allows the phlebologist to never remove their hand from the needle 30 during the blood drawing process, and ensures that none of the blood tubes 16 are lost or misplaced. In addition, by placing the blood tubes 16 in the most convenient location, this eliminates the phlebologist from needing to stretch, twist or otherwise contort themselves to reach for blood tubes 16 that are in an inconvenient location. The convenience of this system 10 also increases the speed of the blood drawing process, reduces accidents and injuries and improves patient satisfaction and confidence. As such, using system 10 improves the safety and efficiency of drawing blood.

Once the phlebologist is done drawing blood, and all of the blood tubes 16 are back in their tube openings 98, the phlebologist grasps the butterfly 32 and withdraws the needle 30 from the patient. Next, the phlebologist moves the shield 36 over the exposed needle 30 to protect from accidental puncture. Next, the phlebologist removes the hub 22 from the main body 56. This is done by pulling hub 22 in the direction opposite of the collar 102 until the frictional engagement between the hub opening 100 and the barrel 24 is overcome. Once separated, the phlebologist simply feeds the tube 28 through slot 104 and the hub 22, tube 28, needle 30, butterfly 32 and shield 36 are ready to be discarded in a sharps container.

This quick, easy and convenient process minimizes a phlebologist's exposure to the contaminated hub 22 and thereby improves safety.

Next, the blood tubes 16 are transported to their desired location in main body 56 and then and there removed from main body 56 and forward on to testing.

Accordingly, from the above discussion it will be appreciated that the improved blood collection wristband and method of use presented offers many advantages over the prior art.

Specifically, the improved blood collection wristband and method of use improves upon the present state of the art; is simple to use; is easy to use; is relatively inexpensive to manufacture; improves safety; reduces repetitive use injuries; conveniently places a hub and a plurality of blood tubes in a single location; is comfortable to wear; holds hubs and blood tubes from various; allows for a butterfly to be removed from the device in a safe and convenient manner; is formed of a minimum number of parts; fits any phlebologist; provides increased convenience; reduces the amount of time needed to draw blood; increases patient comfort; provides a level of reassurance to the patient; has a robust design; allows for quick and easy insertion and withdrawal of blood tubes and hubs; provides the phlebologist with a free hand during the blood drawing process; is durable; is sanitary; has a long useful life; has an intuitive design; among countless other improvements and advantages.

It will be appreciated by those skilled in the art that other various modifications could be made to the device without parting from the spirit and scope of this invention. All such modifications and changes fall within the scope of the claims and are intended to be covered thereby.

What is claimed:

1. A blood collection system, comprising;
a wristband;
the wristband operatively connected to a main body;
the main body having a top side, a bottom side, opposing ends and an opposing front side and back side;
the main body having a plurality of tube openings;
the tube openings extending from the front side of the main body to the back side of the main body;
the main body having a hub opening;
the hub opening extending from the front side of the main body to the back side of the main body;
the hub opening having a slot therein, wherein the slot provides access to the hub opening.

2. The blood collection system of claim 1, wherein the tube openings are generally cylindrical in shape and slightly narrow as they extend from the front side to the back side of the main body.

3. The blood collection system of claim 1, wherein the hub opening is generally cylindrical in shape and slightly narrows as it extends from the front side to the back side of the main body.

4. The blood collection system of claim 1, further comprising a needle holder positioned in the main body.

5. The blood collection system of claim 1, further comprising a needle holder positioned in the main body, wherein the needle holder is generally cylindrical in shape.

6. The blood collection system of claim 1, further comprising a base connected to the main body, wherein the base includes slots that receive the wristband.

7. The blood collection system of claim 1, further comprising a base, wherein the main body is removably detachable from the base so as to facilitate replacement of the main body.

8. The blood collection system of claim 1, wherein the plurality of tube openings includes at least one small tube opening and at least one large tube opening.

9. The blood collection system of claim 1, where a blood tube is positioned within a tube opening.

10. The blood collection system of claim 1, wherein a hub is positioned within the hub opening, the hub having a barrel, a nipple, a tube, a butterfly and a needle.

11. The blood collection system of claim 1, wherein a portion of the top side of the main body angles toward the slot in the hub opening thereby facilitating insertion of a tube into the hub opening.

12. The blood collection system of claim 1, further comprising a collar positioned in an end of the hub opening.

13. The blood collection system of claim 1, further comprising a compressible friction member positioned in one of the hub opening or a tube opening.

14. A method of using a blood collection system, the comprising the steps of;
providing a blood collection system having a wristband, a main body having a plurality of tube openings and a hub opening therein, wherein the hub opening includes a slot that provides access to the hub opening;
placing the blood collection system on the wrist of a phlebologist using the wristband
selecting at least one blood tube;
inserting the at least one blood tube into a tube opening until the blood tube is frictionally held within the tube opening;
selecting a hub, the hub having a barrel, a nipple, a tube and a needle connected thereto;
inserting the tube connected to the hub into the hub opening through the slot in hub opening;
inserting the hub into the hub opening until the hub is frictionally held within the hub opening;
inserting the needle connected to the hub into a patient;
removing a first blood tube from the main body and inserting the blood tube into the hub, thereby drawing blood from the patient.

15. The method of claim 12, wherein a portion of a top side of the main body angles toward the slot in the hub opening thereby facilitating insertion of the tube into the hub opening.

16. The method of claim 12, further comprising the step of inserting the needle into a needle holder positioned in the main body.

17. The method of claim 12, further comprising the step of removing the first blood tube from the hub after the first blood tube has been filled and inserting the filled first blood tube into a tube opening in the main body.

18. The method of claim 12, further comprising the step of removing the hub from the hub opening after drawing blood from the patient by laterally pulling the hub out of the hub opening and removing the tube through the slot in the hub opening.
